(19) United States
(12) Patent Application Publication
(10) Pub. No.: US 2007/0187618 A1
(43) Pub. Date: Aug. 16, 2007

(54) ELECTROSTATIC PARTICLE GETTERING IN AN ION IMPLANTER

(75) Inventor: Robert P. Dolan, Windham, NH (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: IBIS TECHNOLOGY CORPORATION, Danvers, MA (US)

(21) Appl. No.: 11/674,413

(22) Filed: Feb. 13, 2007

Related U.S. Application Data
(60) Provisional application No. 60/773,114, filed on Feb. 13, 2006.

(51) Int. Cl. A61N 5/00

(52) U.S. Cl. 250/492.2

(57) ABSTRACT

Methods and apparatus are disclosed for removing particles from an ion implantation chamber by introducing at least one sacrificial wafer into the implanter and subjecting it to ion implantation. As the sacrificial wafer is exposed to the ion beam, it becomes charged. Particles present in the implantation chamber are then drawn to a charged wafer surface by electrostatic forces. The sacrificial wafer thus serves as a gettering element, attracting and capturing particulates from the surrounding environment.

### RELATED APPLICATION

The present invention claims priority to a provisional application entitled “Electrostatic Particle Gettering in an ION Implanter,” filed on Feb. 13, 2006 and having a Ser. No. 60/773,114. This provisional application is herein incorporated by reference in its entirety.

### BACKGROUND OF THE INVENTION

The technical field of the invention is materials processing by ion implantation and, in particular, the control of contaminants in an ion implantation environment.

Ion implantation is used routinely in many material-processing applications. For example, in SIMOX (separation-by-implantation-of-oxygen) applications, oxygen ions can be implanted into a semiconductor substrate, e.g., a silicon wafer, to generate a buried insulating layer, e.g., SiO₂, through subsequent annealing steps. The successful creation of a buried oxide layer typically requires a long period of exposure to a highly energized beam of oxygen ions. Other implantation protocols for doping, treating or coating of wafers likewise require exposure to charged particles that have been energized by acceleration through an electrostatic potential gradient.

A common problem in the use of ion implantation techniques is that the energized beam of ions not only interacts with the wafer or target but often impinges upon other surfaces of the beam-line chambers or the end station in which the wafer/target is disposed. When the accelerated particles of the beam hit other objects present in the beam-line or end station chambers, the result is often the ejection of material in the form of minute particulates. Despite the typical vacuum conditions, some of the ejected particles are not removed from the chamber but instead settle upon the target and interfere with the ongoing implantation process or otherwise contaminate the processed material.

Despite the typical “clean room” precautions, particulate contaminants can also be introduced into the process environment during the loading and unloading of wafers or as a result of vacuum leaks or material degradation. These particles are likewise disruptive of the implantation process.

In advanced SIMOX processes, e.g., using 300 millimeter wafers, a contaminant level of more than about 300 particles (greater than about 0.2 micrometers in size) per wafer is commonly considered unacceptable. In other SIMOX processes, the acceptable level can range from about 100 to 1000 particles per wafer (ppw). In other processes, such as doping, the constraints on particulate contamination can be even more stringent, e.g. less than 30 ppw.

Conventional approaches to removing particulates from an implantation chamber are typically limited to periodic venting and re-evacuating (purging) of the process chamber or realignment of the ion beam (to reduce undesirable impingements on objects other than the target), followed by the cycling of bare wafers into and out of the end station vacuum chamber (with subsequent recleaning) and/or the processing of wafers that are discarded until an acceptable level of particulates is reached.

There exists a need for better methods and apparatus for gettering particulate contaminants and removing such particles from implantation environments. Techniques that can quickly reduce particle levels and/or avoid wasting of pristine wafers would satisfy a long felt need in the art.

### SUMMARY OF THE INVENTION

Methods and apparatus are disclosed for removing particles from an ion implantation chamber by introducing at least one sacrificial wafer into the implanter and subjecting it to ion implantation. As the sacrificial wafer is exposed to the ion beam, it becomes charged. Particles present in the implantation chamber are then drawn to a charged wafer surface by electrostatic forces. The sacrificial wafer thus serves as a gettering element, attracting and capturing particulates from the surrounding environment.

In one embodiment, the sacrificial wafer can be a conventional silicon wafer with an oxidized surface. Because the surface oxide serves as an insulator, the wafer is quickly charged by the ion beam. Once charged, it attracts and captures particulate contaminants within the chamber. For example, a silicon wafer having a thermally grown oxide on its surface can be used as the sacrificial gettering element. Alternatively, the oxide can be grown by chemical vapor deposition (CVD). Since ion implantation systems are typically designed for automated loading and unloading of wafers of particular sizes, standard and sacrificial wafers can be used interchangeably with little or no handling difficulties.

The sacrificial wafer can have a surface oxide on at least one surface. The thickness of the oxide layer will vary with the particular system requirements but typically will range in thickness from about 100 angstroms to about 10 micrometers, preferably from about 100 nanometers to about 1 micrometer.

### BRIEF DESCRIPTION OF THE DRAWINGS

FIG. 1 schematically illustrates an ion implant apparatus employing electrostatic gettering in accordance with the teachings of the invention;

FIG. 2 is a top view of an exemplary support structure for holding a plurality of sacrificial gettering wafers in an ion beam path in the ion implantation apparatus of FIG. 1; and

FIG. 3 is a cross-sectional view of a sacrificial gettering wafer according to the teachings of the invention.

### DETAILED DESCRIPTION

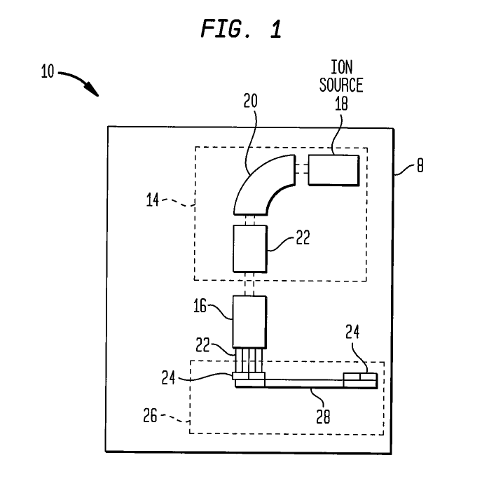

FIG. 1 illustrates an exemplary ion implantation apparatus 10 including a beam delivery assembly 14 and a beam-forming device 16. The apparatus is housed within a chamber 8 that is evacuated to avoid particulate contamination (and to ensure that beam is not dissipated by collisions with gas molecules in the beam path). The beam delivery assembly 14 can include an ion source 18 that generates a beam of ions. The beam delivery assembly 14 can further include an ion analyzer 20, such as a magnetic analyzer, that selects appropriately charged and energized ions. An accelerator 22 accelerates the selected ions to a desired final energy, e.g., about 200 keV, and the beam-forming device 16 shapes the accelerated ions into an ion beam 22 having a selected cross-sectional shape and area. The beam delivery system can further include one or more scanning mechanisms to move the beam across the wafers, if desired.

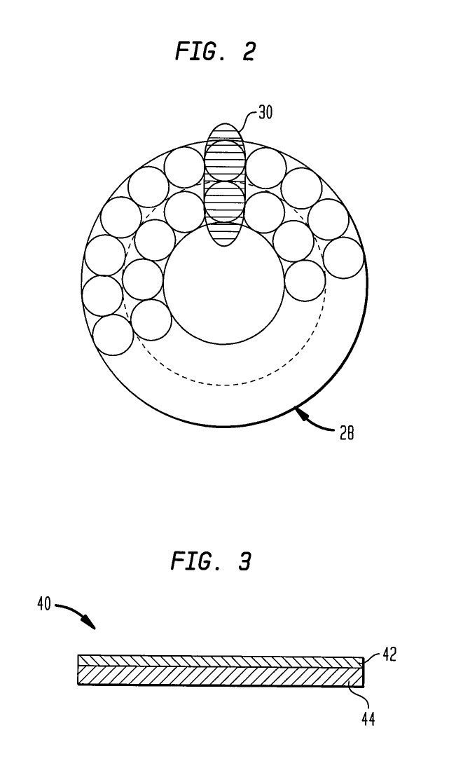

The beam 22 is directed to a plurality of targets 24, e.g., semiconductor wafers, to implant a selected dose of ions therein. In this exemplary embodiment, the targets are disposed in an end-station 26 on a rotating support structure 28. A drive mechanism (not shown) can rotate the support structure to sequentially expose one or more of the wafers 24 to the ion beam 22. For example, as shown in FIG. 2, the exemplary support structure 28 can include an annular platform on which the wafers are held. An exposure zone 30 associated with the beam 22 covers, at each orientation of the support structure 28, two wafers disposed side-by-side along a radial direction of the support structure. Typically the end station 26 is designed to serve as an electrical conduit or ground in order to remove charges that would otherwise build-up on the wafers as a result of ion bombardment.

The exposure zone 30 can, however, extend beyond the cross-sectional area presented by the targets to the beam 22. Hence, a portion of the beam 22 may not be intercepted by the targets and will instead impinge upon the support elements of the end station or other structures within the implantation chamber. This undesired but often unavoidable exposure to the energized ions is a primary cause of particulate contaminants when the beam causes sputtering or ejection of exposed materials.

It should be clear that the exposure zone shown in FIG. 2 is merely illustrative and beam refinements, such as scanning and synchronization, are typically employed to minimize exposure of objects other than the target. Moreover, the depiction of two concentric rings of rotating wafers is also illustrative. As wafer sizes become larger, a single ring (or other arrangements) are commonly employed.

FIG. 3 is a schematic illustration of a sacrificial wafer 40 according to the invention. The dimensions are exaggerated for purposes of illustration. Wafer 40 can be formed of bulk silicon 42 and an thermally grown layer 44 of silicon dioxide. The oxide layer 44 should be sufficiently thick so to serve as a resistive or insulating barrier that permits an electric charge to accumulate on one or more surfaces of the sacrificial wafer. Typically, the field strength of the oxide layer of the sacrificial wafer will be about 3 to about 10 megavolts per centimeter (MV/cm), preferably about 5 to about 10 MV/cm and more preferably greater than about 8 MV/cm in many applications. In many embodiments, the thickness of the oxide layer can be in a range of about 100 angstroms to about 10 micrometers, and preferably in a range of about 100 nanometers to about 1 micrometer.

In use, the present invention can be practiced by introducing a sacrificial substrate into an ion implantation chamber and locating the substrate in the path of an ion beam, activating the ion beam to cause ion impingement on the substrate for about 1 minute to about 1 hour, preferably from about 3 minutes to about 30 minutes until a charge is built up on at least one surface of the substrate sufficient to attract particles present in the chamber, and then removing the sacrificial substrate.

1. A method of cleaning an ion implantation chamber of particles comprising
introducing a sacrificial substrate into an ion implantation chamber and locating the substrate in a path of an ion beam;
activating the ion beam to cause ion impingement on the substrate, and
continuing ion impingement until a charge is built up on at least one surface of the substrate sufficient to attract particles present in the chamber, and
removing the sacrificial substrate.

2. The method of claim 1 wherein the method further comprises deactivating the ion beam, dissipating accumulated charge prior to removing the wafer from the chamber.

3. The method of claim 1 wherein the method further comprises repeating the process of introducing and removing sacrificial wafers into the chamber until an acceptable level of particulate contaminants is achieved.

4. The method of claim 1 wherein the step of introducing a sacrificial wafer further comprises introducing a wafer having a surface oxide layer that exhibits a field strength of about 5 to about 10 MV/cm.

5. The method of claim 1 wherein the step of introducing a sacrificial wafer further comprises introducing a wafer having a surface oxide layer that exhibits a field strength greater than about 8 MV/cm.

6. The method of claim 1 wherein the step of introducing a sacrificial wafer further comprises introducing a wafer having a surface oxide layer with a thickness in the range of about 100 angstroms to about 10 micrometers.

8. The method of claim 1 wherein the step of introducing a sacrificial wafer further comprises introducing a wafer having a surface oxide layer with a thickness in the range of about 100 nanometers to about 1 micrometer.

9. The method of claim 1 wherein the step of activating the ion beam further comprises activating the ion beam to expose the sacrificial wafer to ions for about 1 minute to about 1 hour.

10. The method of claim 1 wherein the step of activating the ion beam further comprises activating the ion beam to expose the sacrificial wafer to ions for about 3 minutes to about 30 minutes.

11. A gettering apparatus for use in cleaning an ion implantation chamber comprising
a sacrificial silicon wafer adapted to be placed in a path of an ion beam; and
at least one oxidized surface of the wafer likewise to be exposed to the beam.

12. The apparatus of claim 11 wherein the sacrificial wafer further comprises a surface oxide layer that exhibits a field strength of about 5 to about 10 MV/cm.

13. The apparatus of claim 11 wherein the sacrificial wafer further comprises a surface oxide layer that exhibits a field strength greater than about 8 MV/cm.

14. The apparatus of claim 11 wherein the sacrificial wafer further comprises a surface oxide layer with a thickness in the range of about 100 angstroms to about 10 micrometers.

15. The apparatus of claim 11 wherein the sacrificial wafer further comprises a surface oxide layer with a thickness in the range of about 100 nanometers to about 1 micrometer.
